(19) United States
(12) Patent Application Publication
(10) Pub. No.: US 2026/0224828 A1
(43) Pub. Date: Aug. 6, 2026

(54) INHALATION DEVICE

(71) Applicant: Merxin Ltd, Norfolk (GB)

(72) Inventors: Graham PURKINS, King's Lynn (GB); Stephen HOWGILL, Leicestershire (GB); Peter David HODSON, Derby (GB); Adam STUART, Pott Row, King’s Lynn (GB); Dylan ANTONIAK, King's Lynn (GB); Adam BARRETT, King's Lynn (GB)

(73) Assignee: Merxin Ltd, King's Lynn, NF (GB)

(21) Appl. No.: 19/148,969

(22) PCT Filed: Jan. 18, 2024
(86) PCT No.: PCT/IB2024/050489

§ 371 Date: Jul. 17, 2025

(30) Foreign Application Priority Data
Jan. 18, 2023 (GB) 2300715.6

(51) Int. Cl. A61M 15/00

(52) U.S. Cl. CPC A61M 15/004; A61M 15/0035; A61M 15/0038; A61M 2202/064; A61M 2206/16

(57) ABSTRACT

A dry powder inhaler includes a tray including a slitting chamber for accommodating a spherocylindrical capsule; a main body configured to receive the tray; the tray and main body moving in use between an open and a closed position; the tray and main body further configured so that as the tray and main body move to the closed position, a capsule in the slitting chamber is squeezed across its length to an oval cross-sectional shape by converging walls; the tray further including a blade adapted to slit or pierce the capsule in the tray when the tray is at or close to the closed position, the blade engaging the capsule surface at a position having a radius of curvature smaller than that of the capsule before the capsule was squeezed.

### TECHNICAL FIELD

The present invention relates to an inhalation device. More particularly, the present invention relates to a dry powder inhaler for dispensing medicament powder from a spherocylindrical capsule. The present invention also relates to a method of loading a spherocylindrical capsule into a dry powder inhaler.

### BACKGROUND

The treatment of respiratory conditions such as asthma and chronic obstructive pulmonary disease (COPD) by using inhaled medicament has been known and practised for a long time. There are many different inhaler types used to deliver medicaments for inhalation. Dry powder inhalers (DPIs) are a popular type of inhaler for this purpose.

The simplest form of DPIs are the so-called “single shot devices”. These use individual gelatin or cellulose capsules containing a single dose of medicament powder, with the inhaler configured so that a user can place a single individual capsule into the inhaler for use. Devices of this type usually have a mechanism operable by a user to pierce the capsule to release medicament powder, the powder from the pierced capsule released into a cavity within the device, and then from there inhaled by the patient.

DPIs of this type have the advantages of low relative manufacturing cost and not requiring environmentally damaging propellant gases. However, creating a mechanism that allows them to operate effectively and consistently can be challenging. Gelatin and cellulose capsules are weak and brittle, and their mechanical properties can change with humidity. This can make them difficult to open in a repeatable way inside an inhaler. This can cause issues with capsule insertion, capsule piercing, inhalation, capsule emptying, and capsule removal.

To attempt to overcome these issues, some existing commercially available DPIs employ sharp metal piercing elements, making them considerably more complex to manufacture and more expensive than they otherwise would need to be. Alternatively, some DPIs use cutting blades to chop off the entire end of a capsule. However, the complete and rapid removal of the capsule end can result in a sudden release of excess medicament powder. If the device is fitted with a downstream powder de-agglomerator, this rapid removal and sudden release of powder can overwhelm the de-agglomerator, leading to a greater hold-up of medicament powder in the inhaler and a less desirable distribution of medicament in the patient's respiratory tract.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

It is an object of the present invention to provide an inhalation device which goes some way to overcoming the abovementioned disadvantages or which at least provides the public or industry with a useful choice.

It is a further object of the invention to provide a method of loading a spherocylindrical capsule into a dry powder inhaler which goes some way to overcoming the abovementioned disadvantages or which at least provides the public or industry with a useful choice.

The term “comprising” as used in this specification and indicative independent claims means “consisting at least in part of”. When interpreting each statement in this specification and indicative independent claims that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

Accordingly, in a first aspect the present invention may broadly be said to consist in a dry powder inhaler for dispensing powder from a spherocylindrical capsule, comprising: a tray comprising a slitting chamber configured to accommodate a spherocylindrical capsule; a main body configured to receive the tray; the tray and main body configured so that they can be moved between an open position where a user can place a capsule in the slitting chamber, and a closed position; the tray and main body further configured so that as the tray and main body are moved from the open to the closed position, a capsule in the slitting chamber is squeezed across its length to an oval cross-sectional shape by converging walls; the tray further comprising at least one blade adapted to slit or pierce a capsule located in the tray when the tray is at or close to the closed position, the blade configured to engage the capsule surface at a position having a radius of curvature smaller than that of the capsule before the capsule was squeezed.

In an embodiment, the converging walls comprise a wedge-shaped ramp that extends inwards towards the slitting chamber, the ramp tapering away from the at least one blade, the main body further configured so that as the tray is moved towards the closed position a portion of the main body contacts a capsule within the tray so that further movement of the tray causes the ramp to engage with the capsule and compress this between the ramp and the main body as the tray is moved to the closed position.

In an embodiment, the tray further comprises a swirl chamber configured to receive the slit capsule from the slitting chamber as the tray is moved to the fully closed position, the swirl chamber configured so that the capsule can rotate within the swirl chamber.

In an embodiment, the dry powder inhaler further comprises at least one air inlet in fluid communication with the swirl chamber so that in use air will enter the swirl chamber tangentially.

In an embodiment, the at least one blade comprises a kite-shaped blade.

In an embodiment, the at least one blade has a trapezoid-type shape with the longest edge inwardly curved.

In an embodiment, the at least one blade comprises a pair of blades, located at or towards each end of the slitting chamber.

In an embodiment, the at least one blade further comprises at least one curved deformation feature configured to deform the capsule walls immediately adjacent to the newly formed slit.

In an embodiment, the at least one blade is configured with two trailing edges, a curved deformation feature attached to each of the trailing edges.

In an embodiment, the dry powder inhaler further comprises at least one substantially circular deformation feature separately positioned downstream of the at least one blade.

In an embodiment, the dry powder inhaler further comprises at least one deformation feature separately positioned downstream of the at least one blade, each of the deformation features triangle-shaped and having an inwardly curved hypotenuse defining a curved ramp.

In an embodiment, the dry powder inhaler further comprises one or more conical piercer elements, each conical piercer element co-located with a blade.

In an embodiment, the at least one blade is integrally formed as part of the tray.

In an embodiment, the at least one blade comprises a metal blade formed separately to the tray.

In an embodiment, the tray and main body are mutually formed so that resistance to relative movement as they are moved from the open position to the closed position substantially increases and then decreases just before the at least one blade contacts the capsule.

in a second aspect the present invention may broadly be said to consist in a method of preparing a spherocylindrical capsule used with a dry powder inhaler for cutting or piercing to dispense powder therefrom, the dry powder inhaler of the type that comprises a tray having a slitting chamber configured to accommodate the capsule during slitting, the method comprising the steps of:
i) placing a capsule in the slitting chamber;
ii) squeezing the capsule across its length to an approximately oval cross-sectional shape by pushing it against the converging walls.

In an embodiment, in the step of squeezing the capsule, the capsule is squeezed by pressing the capsule against converging walls within the inhaler.

In an embodiment, the method of preparing a spherocylindrical capsule comprises the further step of slitting or piercing the capsule at a position having a radius of curvature smaller than that of the capsule before the capsule was squeezed.

In an embodiment, in the step of slitting or piercing the capsule, the capsule is slit with a blade that comprises at least one curved deformation feature configured to deform the capsule walls immediately adjacent to the newly formed slit.

In an embodiment, the method of preparing a spherocylindrical capsule comprises the further step of passing the capsule over a substantially circular deformation feature after cutting or piercing.

In an embodiment, the method of preparing a spherocylindrical capsule comprises the further step of passing the capsule over a triangle-shaped deformation feature after cutting or piercing, the deformation feature having an inwardly curved hypotenuse defining a curved ramp.

In an embodiment, in the step of slitting or piercing the capsule, the capsule is also pierced by at least one conical piercing element.

With respect to the above description then, it is to be realised that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

Further aspects of the invention will become apparent from the following description which is given by way of example only and with reference to the accompanying drawings which show an embodiment of the device by way of example, and in which:

FIG. 1a shows a perspective view of a dry powder inhaler in accordance with a first embodiment of the invention, the inhaler having a body and a slidable tray that moves into and relative to the body, the body forming a mouthpiece with a swirl chamber at the base of the mouthpiece, the inhaler shown in a closed configuration;

FIG. 1b shows a perspective view of the dry powder inhaler of FIG. 1a from a slightly different angle, the inhaler shown in an open configuration;

FIG. 1c shows a perspective view of the underside of the dry powder inhaler of FIGS. 1a and 1b in an open configuration, showing detail of an access hole into which a capsule is inserted in use;

FIG. 1d shows a perspective view from the same angle as FIG. 1c of the underside of the dry powder inhaler of FIGS. 1a-1c in an open configuration, further showing a capsule inserted into the capsule access hole;

FIG. 1e shows a cross-sectional side view of the dry powder inhaler of FIGS. 1a-1d in an open configuration, showing internal detail of the structure of the inhaler;

FIG. 1f shows a cross-sectional side view of the dry powder inhaler of FIGS. 1a-1e from the same angle as FIG. 1e, in an open configuration and further showing a spherocylindrical capsule inserted into the capsule access hole;

FIG. 1g shows a cross-sectional side view of the dry powder inhaler of the preceding figures from the same angle as FIGS. 1e and 1f, the inhaler shown in a partially closed configuration;

FIG. 1h shows a cross-sectional side view of the dry powder inhaler of the preceding figures from the same angle as FIG. 1g, the inhaler shown in a fully closed configuration showing the capsule being ejected from the tray and into the swirl chamber;

FIG. 1i shows a cross-sectional view of the dry powder inhaler of FIGS. 1a-1e from the same angle as FIG. 1f, the inhaler shown in an open configuration and further showing the spherocylindrical capsule in the swirl chamber;

FIG. 1j shows a perspective view from above of the dry powder inhaler in the same configuration as shown in FIG. 1i;

FIG. 1k shows a perspective view of the dry powder inhaler of FIG. 1a, further showing a cover that encloses the inhaler;

FIGS. 2a to 2c provide schematic illustrations of the cutting arrangement of the first embodiment of the invention showing the steps as a capsule is moved further into the inhaler body;

FIG. 3 shows a schematic illustration of a cutting arrangement of a dry powder inhaler according to a second embodiment of the invention;

FIG. 4 shows a schematic illustration of the cutting arrangement of a dry powder inhaler according to a third embodiment of the invention;

FIGS. 5a to 5c show schematic illustrations of different embodiments of capsule opening blades attached to capsule deformation features suitable for use with the embodiments of the dry powder inhaler of the preceding figures;

FIGS. 6a and 6b show schematic illustrations of different embodiments of capsule opening blades positioned in conjunction with capsule deformation features suitable for use with the embodiments of the dry powder inhaler of the preceding figures;

FIGS. 7a and 7b show schematic illustrations of different types of capsule slits that can be obtained using the cutting apparatus of the different embodiments of the invention;

FIG. 8a shows a cross-sectional perspective view of a dry powder inhaler in accordance with a further embodiment of the invention, the inhaler having a slidable tray and lower body component, and slitting blades;

FIG. 8b shows a top view of the slidable tray and lower body component of the dry powder inhaler of FIG. 8a;

FIG. 8c shows a schematic perspective view of a slitting blade of the dry powder inhaler of FIG. 8a;

FIG. 9 shows a schematic perspective view slidable tray in accordance with an embodiment of the invention, the inhaler in this embodiment having kite-shaped slitting blades formed as metal inserts;

FIG. 10 shows a schematic perspective view slidable tray in accordance with an embodiment of the invention having oval-shaped slitting blades formed as metal inserts;

FIG. 11 shows a schematic perspective view slidable tray in accordance with an embodiment of the invention having oval-shaped slitting blades formed as metal inserts and arranged in the floor of the slidable tray;

FIG. 12 shows a schematic perspective view a slidable tray in accordance with a thirteenth embodiment of the invention having semi oval-shaped slitting blades formed as metal inserts.

### DETAILED DESCRIPTION

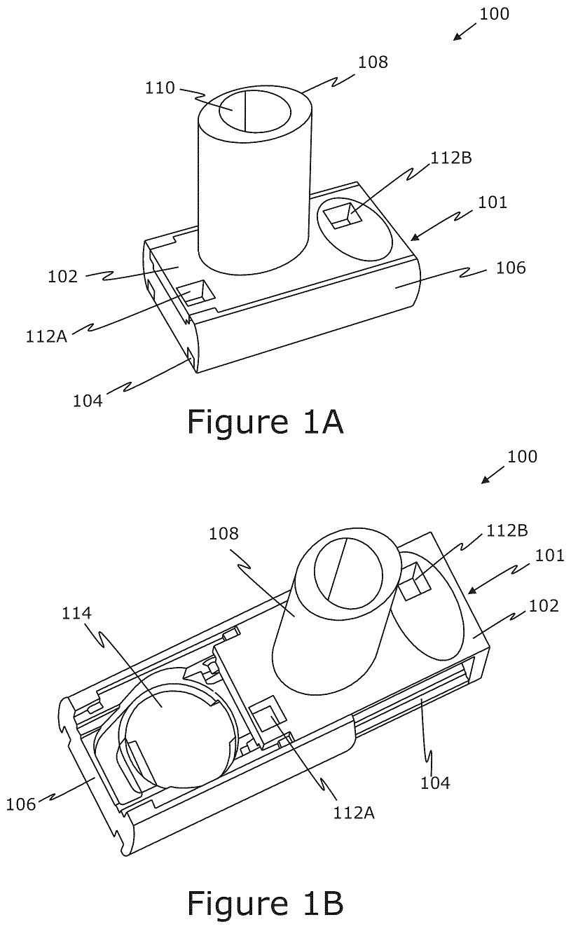

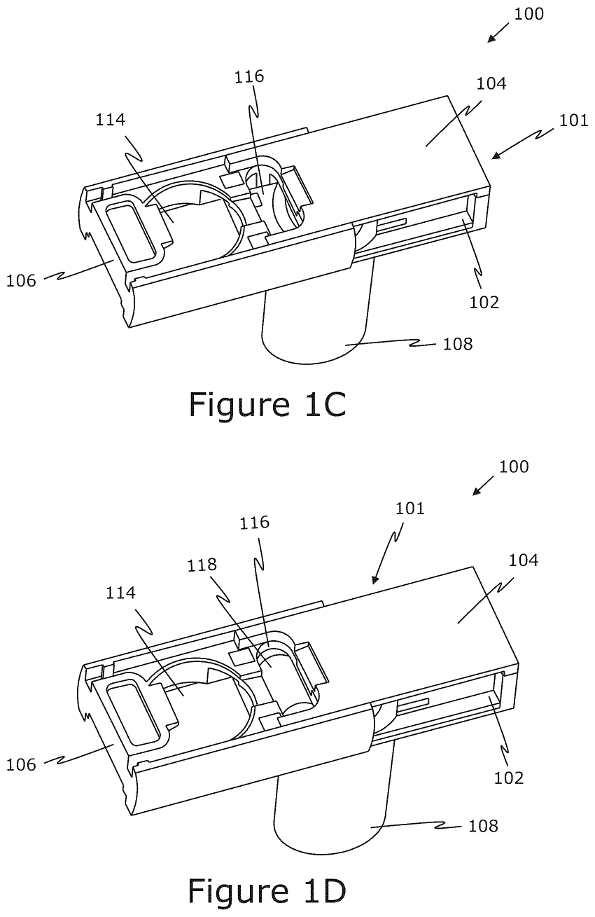

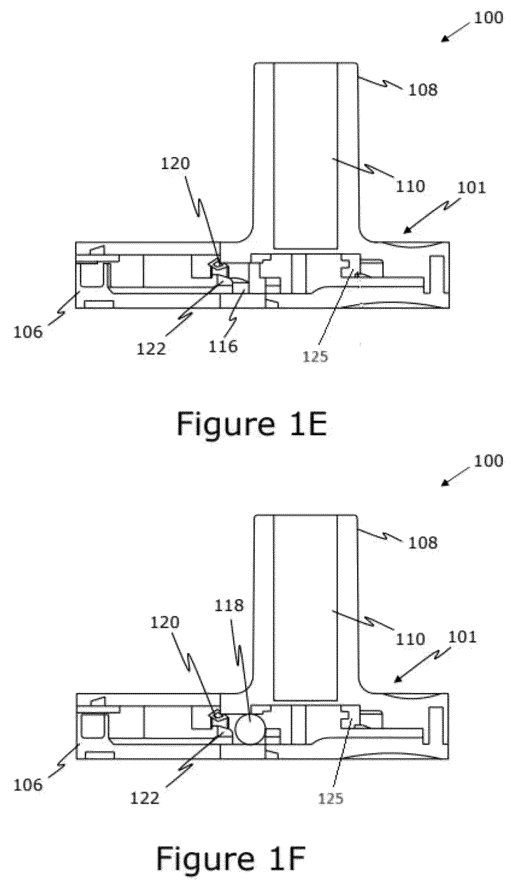

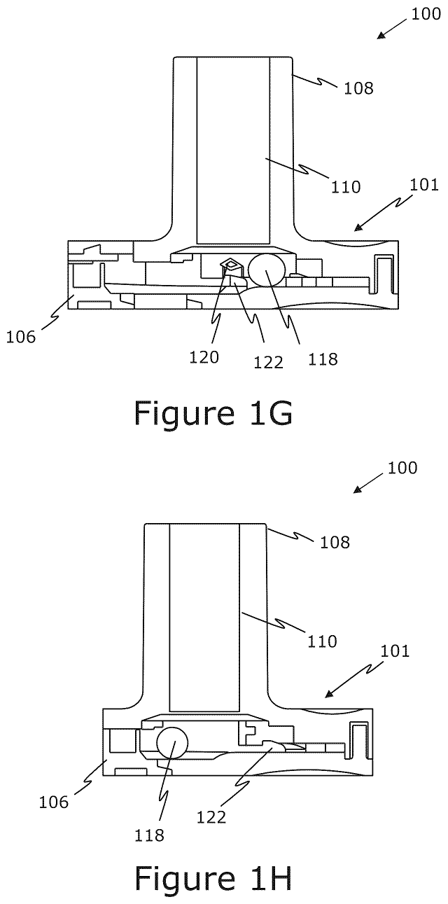

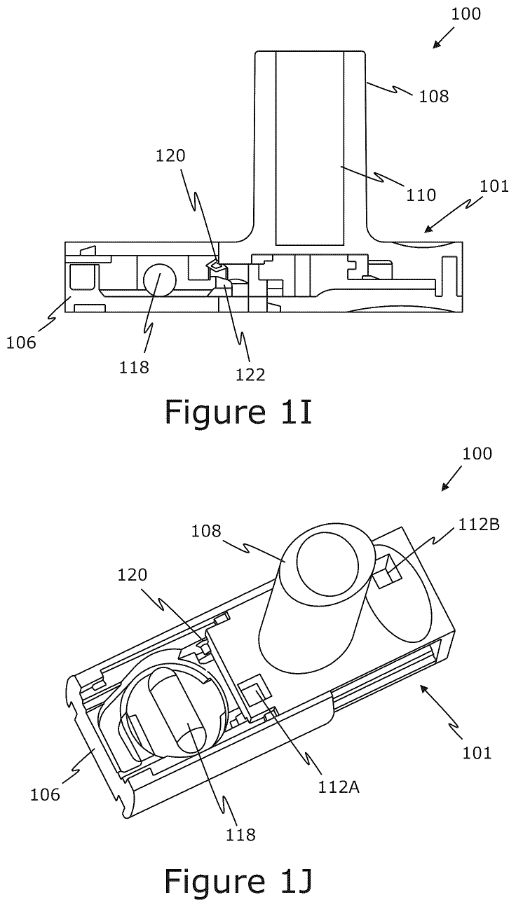

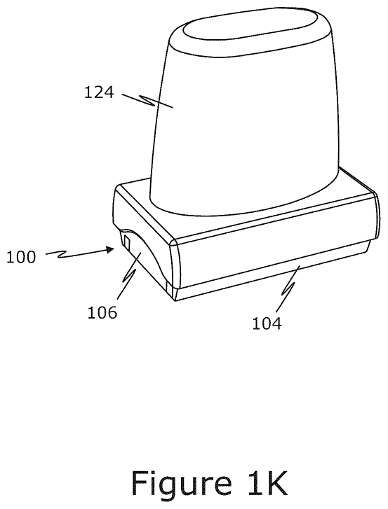

A dry powder inhaler 100 according to a first embodiment of the invention is shown in FIGS. 1a and 1b. The dry powder inhaler 100 comprises two main parts: a body 101 and a slidable tray 106.

In FIG. 1a, the dry powder inhaler 100 is shown in a closed configuration, with the slidable tray 106 fully inserted into the body 101. In FIG. 1b the dry powder inhaler 100 is shown in an open configuration, with the slidable tray 106 slid out of the body 101 such that it is ready to receive a capsule (not shown). In the embodiments described, the dry powder inhaler 100 is intended for use with spherocylindrical gelatine capsules with an outer gelatine shell containing a dry powder medicament formulation.

### Body

The body 101 is formed so as to have an upper body component 102 and a lower body component 104. The lower body component 104 is moulded separately from the upper body component 102 but is rigidly fixed to it during factory assembly. The upper body component 102 comprises a tubular mouthpiece 108 and two square air inlet apertures 112a, 112b. The tubular mouthpiece 108 has an elongated hollow channel 110 that passes through the upper body component 102, the channel 110 having open ends.

### Slidable Tray

As can be seen from FIG. 1b, the slidable tray 106 comprises a tray body that has a large circular aperture defining a swirl chamber 114, which is sized so that in use a capsule in the swirl chamber 114 can rotate within the swirl chamber during use (that is, during inhalation of medicament from the capsule by a user). The side wall of the swirl chamber 114 is defined by the body of the slidable tray 106.

When the dry powder inhaler 100 is in the closed configuration, the upper body component 102 of the body forms a ceiling for the swirl chamber 114. The lower body component 104 of the body defines a floor for the swirl chamber 114 in the closed position. When the dry powder inhaler 100 is in the closed configuration, the open lower end of the elongated hollow channel 110 of the tubular mouthpiece 108 is brought into fluid connection with the swirl chamber 114. It should be noted that a part of the side wall of the swirl chamber 114 is formed by the upper body component 102. This is discussed in more detail below.

FIG. 1c shows the underside of the dry powder inhaler 100 with the inhaler in an open configuration, with the slidable tray 106 slid out of the body 101. As shown in FIG. 1c, the slidable tray 106 further comprises a slitting chamber 116 arranged adjacent to the swirl chamber 114. The slitting chamber 116 has side walls defined by the body of the slidable tray 106 and comprises a capsule-shaped (or “stadium shaped”) access hole in the base of the slidable tray 106. When the dry powder inhaler 100 is in the open configuration, the slitting chamber 116 can receive a spherocylindrical capsule (not shown) through the access hole in the floor of the slidable tray 106. Referring to FIG. 1d, in use, having slid the slidable tray 106 out of the body 101, a user can insert a fresh capsule 118 into the slitting chamber 116.

FIG. 1e shows a cross-sectional view of the dry powder inhaler 100 in an open configuration, with the slidable tray 106 slid out of the body 101. As shown in this figure, the slitting chamber 116 further comprises two kite-shaped slitting blades 120, each of which has a kite-shaped cross-section. The two kite-shaped slitting blades 120 are arranged on opposite sides of the slitting chamber 116 near the top of the swirl chamber 114. It should be noted that only one of the two kite-shaped slitting blades 120 is visible in the cross-sectional view of the dry powder inhaler 100 shown in FIG. 1e and in all other figures from this angle, with the other of the two blades located ‘behind’ the single blade that is in view. The kite-shaped slitting blades 120 are arranged such that the axes of their kite-shaped cross-sections are facing each other. In this example, the slitting blades 120 are co-moulded with the slidable tray 106 during manufacture.

The slidable tray 106 also comprises a wedge-shaped ramp 122 that extends inwards towards the centreline of the slitting chamber in the direction of the two kite-shaped slitting blades 120. In this example, the wedge-shaped ramp 122 is also integrally formed as part of the slidable tray 106.

### Use

FIG. 1f shows a cross-sectional view of the dry powder inhaler 100 of FIG. 1a in an open configuration, with a spherocylindrical capsule 118 inserted into the slitting chamber 116. In practice, this operation would be performed with the tubular mouthpiece 110 pointed downwards as in FIG. 1d, so that the capsule 118 can be dropped into the slitting chamber 116 under gravity. Once inserted into the slitting chamber 116, the capsule 118 sits against the wedge-shaped ramp 122 as shown in FIG. 1f.

Once the capsule is inserted into the slitting chamber 116, the user then slides the slidable tray 106 back into the body 101, as shown in FIG. 1g. The two kite-shaped slitting blades 120 and wedge-shaped ramp 122 carry the capsule 118 into the body 101, in the direction of movement of the slidable tray 106 back into the body 101, such that the capsule passes beneath the elongated hollow channel 110 of the tubular mouthpiece 108. By this point in the operation, the capsule 118 has not met any significant resistance, and so has not been deformed or penetrated in any way.

As mentioned above, part of the side wall of the swirl chamber 114 is formed by the upper body component 102, rather than by the slidable tray 106. This element (the part of the side wall) is shown in FIGS. 1f, 1g, and 1h, and is numbered as side wall 125 This means that as the tray is pushed from the open to the closed position, the capsule 118 contacts the side wall element 125 and, as the tray continues to move towards the closed position, is then effectively pushed towards the swirl chamber 114 by the side wall element 125 as the slidable tray 106 is slid into the body 101 (in actuality, the capsule 118 remains stationary as the tray continues to move around and underneath it, with the swirl chamber 114 being pushed towards the stationary capsule).

Therefore, as the slidable tray 106 is slid completely back into the body 101 as shown in FIG. 1h, at the point at which the capsule 118 is located right up to the edge of the swirl chamber 114 (at or nearly in the fully closed position for the inhaler 100), the wedge-shaped ramp 122 on the tray contacts and then lifts the capsule 118 against the roof of the swirl chamber 114 (defined by the upper body component 102). This squashes the cross-section of the capsule 118 into an oval shape, and the kite-shaped slitting blades 120 travel through the now-constrained ends of the capsule 118, forming two slits through which the medicament powder can exit the capsule. The capsule 118 is then ejected into the swirl chamber 114, as shown in FIG. 1h, where it is free to move (e.g. rotate) within the swirl chamber 114. The kite-shaped slitting blades 120 pass into recesses (not shown) located in the side wall of the swirl chamber 114, such that the kite-shaped slitting blades 120 are kept out of the way of the slit capsule 118 once it is located in the swirl chamber 114.

Once this operation is completed, the patient inhales through the open top of the tubular mouthpiece 108, drawing air into the dry powder inhaler 100 via the two square air inlet apertures 112A, 112B-these are configured so that airflow passageways lead from the apertures 112A, 112B tangentially into the swirl chamber 114. The capsule 118 is shorter in length than the internal diameter of the swirl chamber 114, so the resulting tangential airflow thus causes the capsule 118 to move within the swirl chamber 114. Due to the size and shape of the capsule and chamber, this movement tends to be a partially rotational and partially chaotic tumbling motion.

This motion of the capsule 118, in conjunction with the airflow, flings and sucks medicament powder out from the capsule 118 via the slits, creating a powder aerosol that is drawn out from the chamber 114, along the elongated hollow channel 110 of the tubular mouthpiece 108, and then out of the dry powder inhaler 100 into the patient's respiratory tract.

Optionally, the dry powder inhaler 100 may comprise a powder deagglomeration means between the swirl chamber 114 and the exit of the tubular mouthpiece 108. This powder deagglomeration means may be in the form of a capsule retaining grid at the lower end of the tubular mouthpiece 108 (i.e. directly above the swirl chamber 114), but other arrangements are possible.

As shown in FIG. 1i, in order to discard the spent capsule 118, the patient pulls the slidable tray 106 back out of the body 101 again (i.e. back to the open configuration) after finishing their inhalation or inhalations. FIG. 1J provides a perspective view of the dry powder inhaler 100 shown in FIG. 1I. As shown in both FIGS. 1I and 1J, this brings the capsule 118 to a point at which it can fall out of the swirl chamber 114 of the dry powder inhaler 100. It should be noted that the capsule 118 does not need to pass back over the kite-shaped slitting blades 120 at any point, thereby avoiding the risk of fragments being cut from it.

The patient can then slide the slidable tray 106 back into the body 101 and place the inhaler 100 within a protective cover 124 for storage as shown in FIG. 1k, until the next occasion they need to use the dry powder inhaler 100. The protective cover 124 sits over the dry powder inhaler 100 and encloses the tubular mouthpiece 108 as well as the two square air inlet apertures 112a, 112b, thereby protecting them from the ingress of dirt, dust, and unwanted small objects.

In the dry powder inhaler 100 according to the first embodiment of the invention, as depicted by FIGS. 1a to 1k, the wedge-shaped ramp 122 and roof of the swirl chamber 114 as defined by the upper body component 102 together provide capsule squeezing surfaces that compress the capsule 118 into an oval cross-sectional shape at the point of capsule slitting.

Thus, the kite-shaped slitting blades 120 encounter a capsule surface having a radius of curvature smaller than that of the capsule 118 before it was compressed. This confers an important advantage: because gelatine capsules are weak and easily distorted, they tend to crush and split untidily rather than to slit neatly when sliced. By compressing them vertically, the surface (i.e. wall) of the capsule 118 presented to the slitting blades 120 is reduced in radius of curvature locally, which effectively makes it stronger and more rigid.

This means that the capsule 118 is better able to resist unwanted crushing and deformation. Instead, the capsule 118 is more readily slit neatly by the slitting blades 120 as they pass through the ends of the capsule 118. In other words, the capsule squeezing surfaces of the dry powder inhaler 100, which reduce the local radius of curvature of the surfaces of the capsule 118 where it is to be slit, improve the quality and consistency of the openings made in the capsule 118, and that in turn improves the consistency of powder release and reduces the risk of gelatine debris generation.

It should also be noted that the dry powder inhaler 100 provides a separate entry port (the capsule-shaped access hole of the slitting chamber 116) and an exit port (swirl chamber 114) for the capsule 118. This is in contrast to for example the inhaler disclosed in U.S. Pat. No. 8,677,992. This can help to prevent a situation where a patient is potentially confused over whether a capsule 118 located in the dry powder inhaler 100 has been used and forgotten or is fresh and unused.

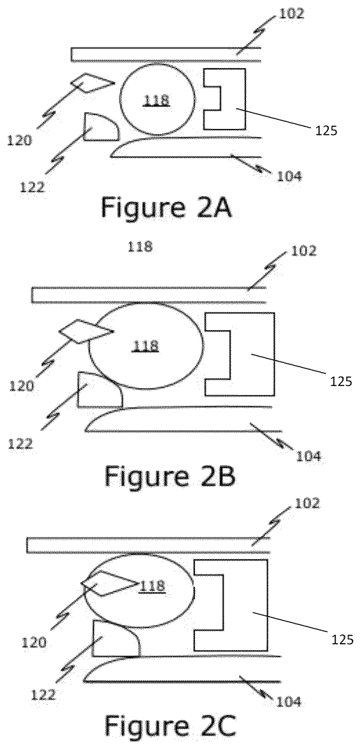

FIGS. 2a to 2c provide a schematic representation of the cutting arrangement of the dry powder inhaler 100 according to the first embodiment of the invention, wherein the wedge-shaped ramp 122 in the floor of the slidable tray 106 lifts and squeezes the capsule 118 as the slidable tray 106 is slid into the body 101 of the dry powder inhaler 100. In FIG. 2a, the relative positions of the features are like those of FIG. 1g, with the capsule 118 not yet in contact with the leading edge of the two kite-shaped slitting blades 120 or the side wall element 125, and not yet compressed vertically.

Referring to FIG. 2b, as the slidable tray 106 is slid further back into the body 101, the capsule 188 contacts the side wall element 125 and stops moving, and the wedge-shaped ramp 122 and two kite-shaped slitting blades 120 make contact with the capsule 118 on the other side of the capsule 188 (the left-hand side in the figure). The capsule 188 therefore becomes constrained, and the wedge-shaped ramp 122 starts to squeeze the cross-section of the capsule 118 into an oval shape. The slitting blades 120 therefore encounter regions of the surface of the capsule 118 that have a radius of curvature smaller than those of the capsule 118 before it was compressed. Such regions of reduced radius of curvature are effectively stronger and more rigid and are thus better able to withstand the tendency to crush and deform. Slitting is thus ‘cleaner’, more reproducible, and less prone to generate loose gelatine debris from the capsule 118.

Referring now to FIG. 2c, once the slidable tray 106 is slid even further back into the body 101, the capsule 118 is compressed even further, reducing its radius of curvature even further as it passes the slitting blades 120. Thus, the tendency of the capsule 118 to crush and deform as the slitting blades 120 pass through the capsule 118 is even further reduced. As mentioned above, this allows ‘clean’ slitting of the ends of the capsule 118 in the dry powder inhaler 100, an operation that is otherwise difficult to perform consistently, due to the readily crushed nature of gelatine capsules. Even when presented with high-quality blades as sharp and thin and hard as scalpel blades, these types of capsules can be difficult to slit ‘cleanly’. This operation is therefore potentially more difficult when using lower-quality mass-produced moulded plastic blades, which need to be made sufficiently thick to have strength and rigidity, and there is therefore a greater chance of having the problem of unwanted, inconsistent crushing or deformation. The dry powder inhaler 100 of the present invention is advantageously configured to help overcome these issues.

Moreover, compression of the capsule 118 by the dry powder inhaler 100 is performed “invisibly” to the user as the slidable tray 106 is slid back into the body 101-the user cannot see the compression taking place. This avoids the need for example for the patient to have to try to squeeze the capsule 118 into a slot narrower than its diameter, and thus helps to avoid difficulty or confusion. Nevertheless, alternative embodiments of the present invention may employ a narrow capsule-shaped cavity (i.e. capsule entry port) as the means to compress the capsule as it reaches the slitting blades.

### Variations and Alternative Embodiments

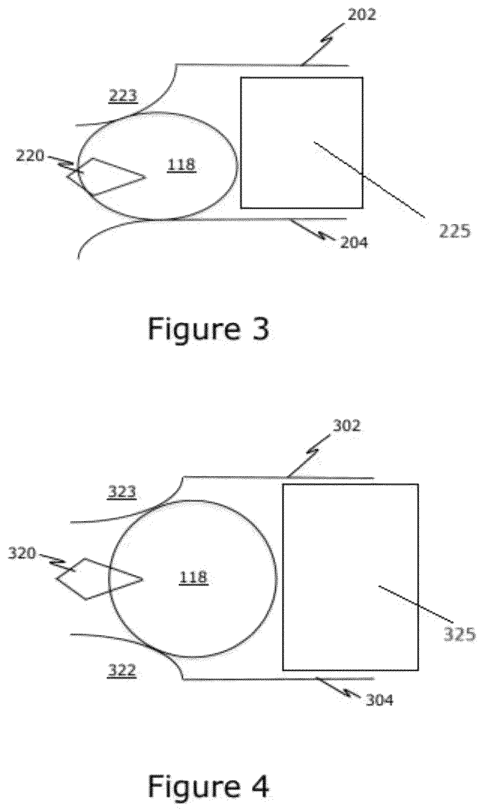

FIG. 3 provides a schematic representation of the cutting arrangement of a dry powder inhaler 200 according to a second embodiment of the invention. The dry powder inhaler 200 is substantially similar to the inhaler 100 (and similar numbering is used for similar components). However, in this embodiment the upper body component 202 provides a wedge-shaped ramp 223, and the capsule 118 is compressed downwards towards the lower body component 204 rather than upwards towards the upper body component 202. The blades are also located on the main body rather than the tray. In use, movement of the tray moves the capsule 118 and compresses the capsule 118 against the wedge-shaped ramp 223, as shown. The tray wall 225 as shown in the figure moves from right to left when moving from the ‘open’ to the ‘closed’ position, in contrast with the first embodiment as shown in FIGS. 2a to 2c, where movement of the tray is from left to right and the main body remains stationary.

FIG. 4 provides a schematic representation of the cutting arrangement of a dry powder inhaler 300 according to a third embodiment of the invention. The dry powder inhaler 300 is substantially similar to the inhaler 100 (and similar numbering is used for similar components). However, in this embodiment the upper body component 302 provides a wedge-shaped ramp 323 and the lower body component 304 also provides a wedge-shaped ramp 322, and the capsule 118 is compressed between the ramps 322, 323 as shown in FIG. 4. The blades are also located on the main body rather than the tray. In use, movement of the tray moves the capsule 118 and compresses the capsule 118 against the wedge-shaped ramps 322, 323, as shown. The tray wall 325 as shown in the figure moves from right to left when moving from the ‘open’ to the ‘closed’ position, in contrast with the first embodiment as shown in FIGS. 2a to 2c, where movement of the tray is from left to right and the main body remains stationary.

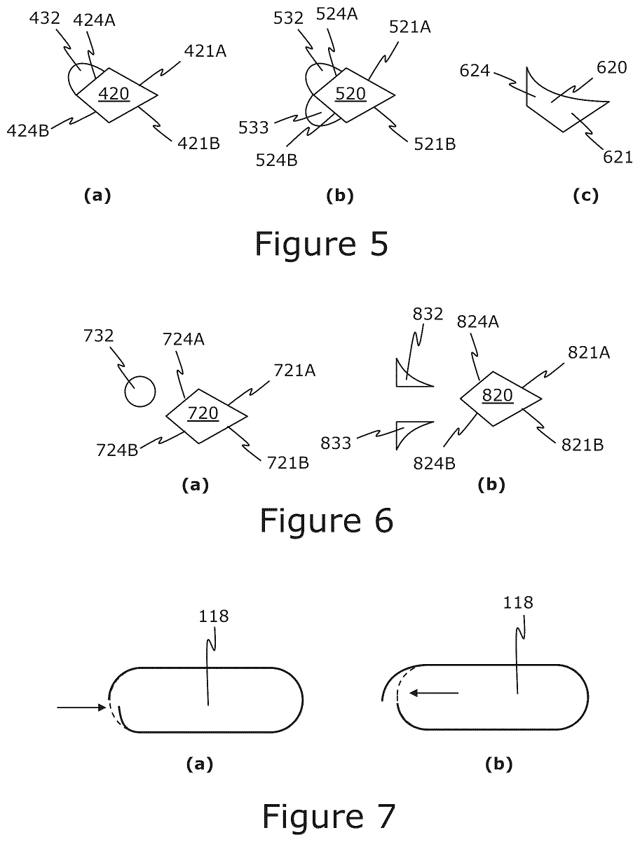

FIG. 5(a) to (c) illustrate various embodiments of capsule opening blades and associated with capsule deformation features that in use act to help open the slits that have been immediately previously created in a capsule by the capsule opening blades. The capsule opening blades and capsule deformation features in these embodiments can be used with dry powder inhalers that are substantially similar or identical to the inhaler 100, and similar numbering is used for similar components.

FIGS. 5(a) shows a kite-shaped slitting blade 420 having two leading edges 421A, 421B for slitting a capsule. The kite-shaped slitting blade 420 also has first and second trailing edges 424A, 424B. A curved deformation feature 432 is attached to the first trailing edge 424A. The curved deformation feature 432 is arranged to open the slit in the capsule by a pre-determined amount, or to otherwise deform the capsule walls immediately adjacent to the newly formed slit.

FIG. 5(b) shows a kite-shaped slitting blade 520 having two leading edges 521A, 521B for slitting a capsule. The kite-shaped slitting blade 520 also has first and second trailing edges 524A, 524B. First and second curved deformation features 532, 533 are attached to the first and second trailing edges 524A, 524B respectively. The curved deformation features 532, 533 are arranged to open the slit in the capsule by a pre-determined amount, or to otherwise deform the capsule walls immediately adjacent to the newly formed slit.

FIG. 5(c) shows a slitting blade 620 have a trapezoid-type shape, in which the longest edge is inwardly curved. The slitting blade 620 has a leading portion 621 for slitting a capsule. The slitting blade 620 also has a trailing portion 624 arranged to open the slit in the capsule by a pre-determined amount, or to otherwise deform the capsule walls immediately adjacent to the newly formed slit.

FIGS. 6(a) and (b) illustrate embodiments of capsule opening blades positioned in conjunction with capsule deformation features that open the slits created in a capsule by the blades. FIG. 6(a) shows a kite-shaped slitting blade 720 having two leading edges 721A, 721B for slitting a capsule. The kite-shaped slitting blade 720 also has first and second trailing edges 724A, 724B. A circular deformation feature 732 is separately positioned downstream near to the first trailing edge 724A. The circular deformation feature 732 is arranged to open the slit in the capsule by a pre-determined amount, or to otherwise deform the capsule walls immediately adjacent to the newly formed slit.

FIG. 6(b) shows a kite-shaped slitting blade 820 having two leading edges 821A, 821B for slitting a capsule. The kite-shaped slitting blade 820 also has first and second trailing edges 824A, 824B. First and second deformation features 832, 833 are positioned so as to be slightly separate to the blades, downstream of the blades near to the first and second trailing edges 824A, 824B respectively. Each of the first and second deformation features 832, 833 is triangle-shaped and has an inwardly curved hypotenuse (i.e. thereby defining a curved ramp). The first and second deformation features 832, 833 are arranged to open the slit in the capsule by a pre-determined amount, or to otherwise deform the capsule walls immediately adjacent to the newly formed slit.

Referring to FIGS. 7(a) and (b), for all embodiments, the blades and deformation features are configured so that when the capsule is slit, the deformation to the newly slitted capsule walls should be suitably great as to allow for the formation of a finite opening in the capsule and for plastic deformation so that the walls do not rapidly spring closed again. FIG. 7(a) shows that this can be achieved by controllably deforming the capsule wall inwards from the slit. FIG. 7(b) shows that this can be achieved by controllably deforming the capsule walls outwards from the slit. Note that in both FIG. 7(a) and (b), the slit end of the capsule 118 has been left with an “over-bite”. Embodiments of the present invention may alternatively be suitably arranged to form an “under-bite”, however.

In some embodiments, both ends of the capsule may be slit. In some cases, both slit ends may be controllably deformed. For example, both ends may be given controlled “over-bites”. Alternatively, one end may be given an “over-bite” and one end may be given an “under-bite”. In all cases, features of the dry powder inhalers according to the invention may suitably be arranged to form controlled openings in the capsule to achieve satisfactory and consistent powder release rates and to avoid capsule fragments being formed unnecessarily. It is generally desirable that capsule slits are sufficiently open that all the powder can emerge from the capsule during the duration of one or more patient inhalations.

Gelatine capsules have different behaviour characteristics when pierced or slit quickly or slowly. To reduce the influence of this speed dependence, the embodiments of dry powder inhalers according to the present invention may comprise resistance features. Such resistance features may advantageously create a controlled degree of resistance to movement of the capsule towards a position within the dry powder inhaler where it is to be opened by slitting or piercing or similar.

Conveniently, this may be provided by “bump” features associated with movement of the capsule towards the slitting blades. For example, the slidable tray may have features that pass over “bump” features as it slides into the body of the dry powder inhaler, offering a short period of increased resistance to its movement. This can have the effect of making the speed of movement of the tray rather independent of the patient's actions for the distance of movement immediately after the “bump”. In other words, the sudden reduction of movement resistive force on the slidable tray immediately after the “bump” is passed ensures that the slidable tray moves at a rapid and relatively patient-independent speed. By relatively positioning the “bumps” and the slitting blades appropriately, this ensures that the capsule contacts the slitting blades at consistent and relatively fast speeds. This ensures more accurate slitting of the ends of the capsule, with less tendency for random and uncontrolled capsule deformation. In some embodiments, the converging walls of the dry powder inhaler may provide the “bump” function.

Although integrally injection-moulded plastic slitting blades and/or associated features may be preferred for reasons of cost, metal slitting blades may alternatively or additionally be employed. Piercers rather than slitting blades may alternatively or additionally be employed. One or both ends of each capsule may be opened by the dry powder inhaler. Other positions on the capsule may be slit or pierced, as well as or instead of the ends. Slitting may be symmetrical at each end of the capsule, or it may be asymmetric. Slitting and/or piercing may be conducted on or off the capsule's centreline. One end of the capsule may be slit, and the other end pierced. One end may be opened slightly before the other.

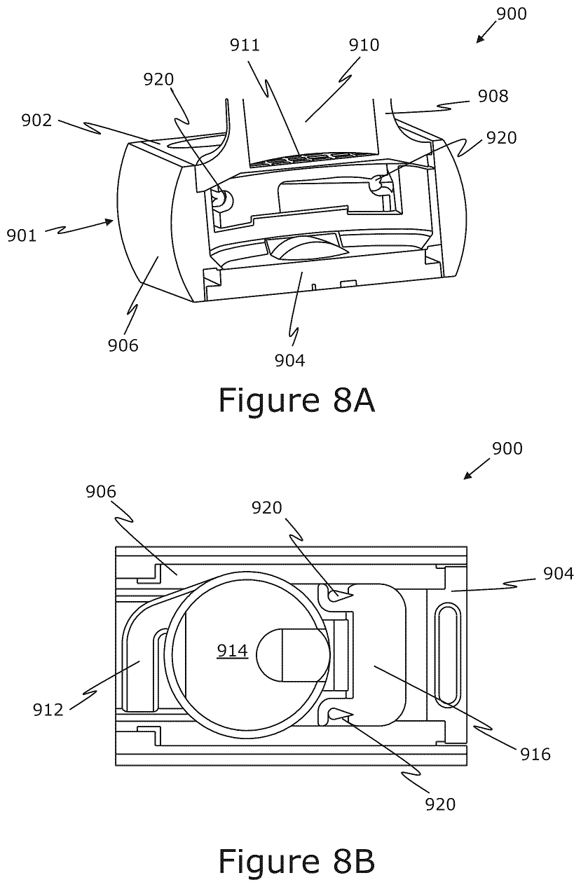

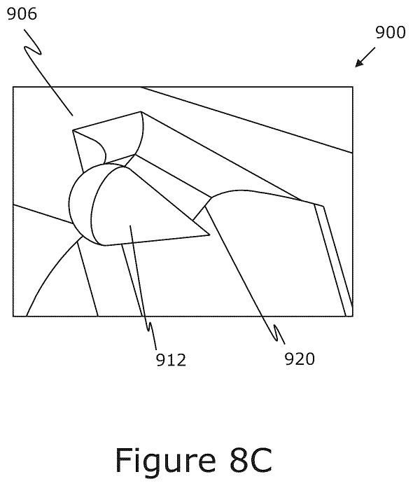

Referring now to FIG. 8a, a dry powder inhaler 900 in accordance with an embodiment of the invention comprises a body 901 and a slidable tray 906. The body 901 is formed from an upper body component 902 and a lower body component 904. The upper body component 902 comprises a tubular mouthpiece 908. The tubular mouthpiece 908 has an elongated hollow channel 910, both being only partially shown in FIG. 8a. The upper body component 902 comprises a capsule retaining grid 911 that covers the start of the elongated hollow channel 910. In FIG. 8a, the dry powder inhaler 900 is shown in a closed configuration, whereby the slidable tray 906 is fully inserted into the body 901.

FIG. 8b provides a top view of the slidable tray 906 fully engaged with the lower body component 904 (i.e. a view which could be seen if the upper body component 902 was disconnected and removed from the lower body component 904). As shown in FIG. 8b, the slidable tray 906 comprises a capsule-shaped cavity 916, which defines a capsule access hole for receiving a spherocylindrical capsule (not shown), and a large circular aperture defining a swirl chamber 914. Also visible in FIG. 8b is an air passage 912, which fluidly connects to an air inlet aperture in the upper body component 902 (not shown).

FIG. 8c provides an enlarged view of the slitting blades 920 of the dry powder inhaler 900, in which conical piercer elements 921 are formed at the outer ends of the slitting blades 920. The addition of the conical piercer elements 921 to the slitting blades 920 causes a somewhat greater, and less temporary, opening up of the slits formed in the capsule ends than using only blades, thereby enhancing (in a controlled way) the release of the powdered medicament dosage supplied in the capsule. It has been found that the conical piercer elements 921 leave open gouges through the capsules, and yet they form no visible capsule debris. The slitting blades 920 and conical piercer elements 921 can be provided in a way that still allows easy injection moulding of the slidable tray 906, without a need for side-actions on the injection moulding tooling used to form the slidable tray 906.

As shown in FIG. 8a, the conical piercer elements 921 advance into slots within the upper body component 902 as they pass through the capsule's walls during capsule slitting. This means that the upper body component 902 provides good support to the regions of the capsule's walls close to the positions that will be slit. In other words, effectively shrouding the slitting blades 920 and conical piercer elements 921 in this way provides good support to the capsule locally, thus holding it more rigidly while the blades pass through it. This produces “cleaner” and more consistent openings in the capsule.

The extra resistance to local capsule deformation that is provided by such shrouding of the slitting blades 920 and conical piercer elements 921 has also been found beneficial in other embodiments, such as those of the general types illustrated in FIGS. 6(a) and (b). For example, slitting blades having a “kite” cross-section, as illustrated in FIGS. 6(a) and (b), each in association with a following square wedge to push the slit more open, have been found to benefit from such close shrouding of the slitting blades.

It should be noted that the support for the local areas of the capsule walls herein described, which provides resistance to capsule movement and deformation during slitting, is inventively different to that disclosed in for example U.S. Pat. No. 8,677,992, referred to above. In the device described in U.S. Pat. No. 8,677,992, the capsule is simply supported in front and behind, but no support is provided immediately around the regions of the capsule walls through which the slitting blades must pass. Conversely, as disclosed herein, this embodiment of the invention provides much closer support to the regions of the capsule wall that are to be slit. This has been found to improve the nature and consistency of the slits produced.

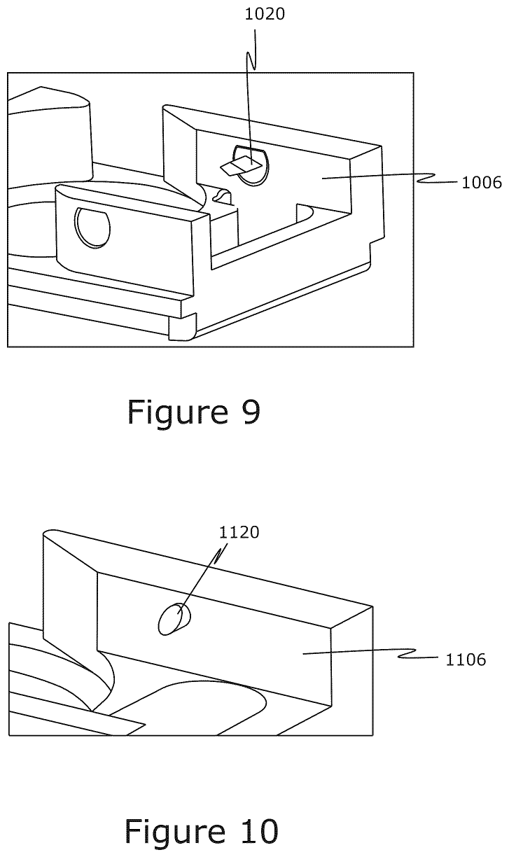

Referring to FIG. 9, a slidable tray 1006 having kite-shaped slitting blades 1020 formed as metal inserts that can be inserted into the plastic moulded slidable tray 1006 (rather than being formed from moulded plastic) is shown. Notably, the benefit of forming the slitting blades as metal inserts is that they are more resistant to wear and blunting through the life of the dry powder inhaler, giving more consistent piercing geometry. Moreover, providing the slitting blades as metal inserts may also increase the flexibility of design choice of the size, shape, and location of the slitting blades, to allow for tuning of the piercing geometry. Such metal inserts could be over-moulded, or inserted during assembly.

FIG. 10 shows a slidable tray 1106 having oval-shaped slitting blades 1120 formed as metal inserts.

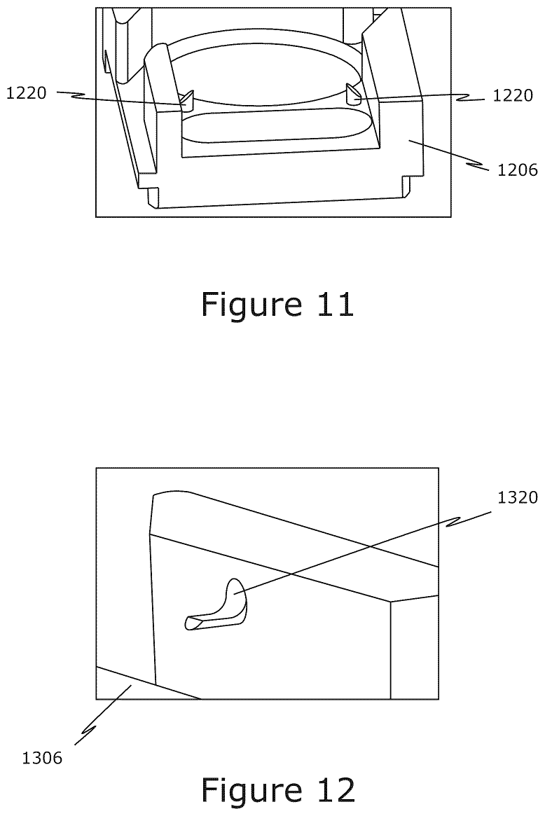

FIG. 11 shows a slidable tray 1206 having oval-shaped slitting blades 1220 formed as metal inserts and arranged in the floor of the slidable tray 1206.

FIG. 12 shows a slidable tray 1306 having semi-oval-shaped slitting blades 1320 formed as metal inserts.

1. A dry powder inhaler for dispensing powder from a spherocylindrical capsule, comprising:
a tray comprising a slitting chamber configured to accommodate a spherocylindrical capsule;
a main body configured to receive the tray;
the tray and main body configured so that they can be moved between an open position where a user can place a capsule in the slitting chamber, and a closed position;
the tray and main body further configured so that as the tray and main body are moved from the open to the closed position, a capsule in the slitting chamber is squeezed across its length to an oval cross-sectional shape by converging walls;
the tray further comprising at least one blade adapted to slit or pierce a capsule located in the tray when the tray is at or close to the closed position, the blade configured to engage the capsule surface at a position having a radius of curvature smaller than that of the capsule before the capsule was squeezed.

2. The dry powder inhaler as claimed in claim 1 wherein the converging walls comprise a wedge-shaped ramp that extends inwards towards the slitting chamber, the ramp tapering away from the at least one blade, the main body further configured so that as the tray is moved towards the closed position a portion of the main body contacts a capsule within the tray so that further movement of the tray causes the ramp to engage with the capsule and compress this between the ramp and the main body as the tray is moved to the closed position.

3. The dry powder inhaler as claimed in claim 2 wherein the tray further comprises a swirl chamber configured to receive the slit capsule from the slitting chamber as the tray is moved to the fully closed position, the swirl chamber configured so that the capsule can rotate within the swirl chamber.

4. The dry powder inhaler as claimed in claim 3 further comprising at least one air inlet in fluid communication with the swirl chamber so that in use air will enter the swirl chamber tangentially.

5. The dry powder inhaler as claimed in claim 1 wherein the at least one blade comprises a kite-shaped blade.

6. The dry powder inhaler as claimed in claim 1 wherein the at least one blade has a trapezoid-type shape with the longest edge inwardly curved.

7. The dry powder inhaler as claimed in claim 1 wherein the at least one blade comprises a pair of blades, located at or towards each end of the slitting chamber.

8. The dry powder inhaler as claimed in claim 5 wherein the at least one blade further comprises at least one curved deformation feature configured to deform the capsule walls immediately adjacent to the newly formed slit.

9. The dry powder inhaler as claimed in claim 8 wherein the at least one blade is configured with two trailing edges, a curved deformation feature attached to each of the trailing edges.

10. The dry powder inhaler as claimed in claim 5 further comprising at least one substantially circular deformation feature separately positioned downstream of the at least one blade.

11. The dry powder inhaler as claimed in claim 5 further comprising at least one deformation feature separately positioned downstream of the at least one blade, each of the deformation features triangle-shaped and having an inwardly curved hypotenuse defining a curved ramp.

12. The dry powder inhaler as claimed in claim 1 further comprising one or more conical piercer elements, each conical piercer element co-located with a blade.

13. The dry powder inhaler as claimed in claim 1 wherein the at least one blade is integrally formed as part of the tray.

14. The dry powder inhaler as claimed in claim 1 wherein the at least one blade comprises a metal blade formed separately to the tray.

15. The dry powder inhaler as claimed in claim 1 wherein the tray and main body are mutually formed so that resistance to relative movement as they are moved from the open position to the closed position substantially increases and then decreases just before the at least one blade contacts the capsule.

16. A method of preparing a spherocylindrical capsule used with a dry powder inhaler for cutting or piercing to dispense powder therefrom, the dry powder inhaler of the type that comprises a tray having a slitting chamber configured to accommodate the capsule during slitting, the method comprising the steps of:
i) placing a capsule in the slitting chamber;
ii) squeezing the capsule across its length to an approximately oval cross-sectional shape by pushing it against the converging walls.

17. The method of preparing a spherocylindrical capsule as claimed in claim 16 wherein in the step of squeezing the capsule, the capsule is squeezed by pressing the capsule against converging walls within the inhaler.

18. The method of preparing a spherocylindrical capsule as claimed in claim 16 comprising the further step of slitting or piercing the capsule at a position having a radius of curvature smaller than that of the capsule before the capsule was squeezed.

19. The method of preparing a spherocylindrical capsule as claimed in claim 18 wherein in the step of slitting or piercing the capsule, the capsule is slit with a blade that comprises at least one curved deformation feature configured to deform the capsule walls immediately adjacent to the newly formed slit.

20. The method of preparing a spherocylindrical capsule as claimed in claim 18 comprising the further step of passing the capsule over a substantially circular deformation feature after cutting or piercing.

21-22. (canceled)
